(19) United States
(12) Patent Application Publication
(10) Pub. No.: US 2026/0272396 A1
(43) Pub. Date: Sep. 17, 2026

(54) X-RAY CT APPARATUS, IMAGE PROCESSING APPARATUS, AND IMAGE PROCESSING METHOD

(71) Applicant: CANON MEDICAL SYSTEMS CORPORATION, Otawara-shi (JP)

(72) Inventors: Takuo NEGISHI, Nasushiobara (JP); Sho SASAKI, Ota (JP); Kosuke ARITA, Kawasaki (JP)

(73) Assignee: CANON MEDICAL SYSTEMS CORPORATION, Otawara-shi (JP)

(21) Appl. No.: 19/560,866

(22) Filed: Mar. 9, 2026

(30) Foreign Application Priority Data
Mar. 12, 2025 (JP) 2025-039739

(51) Int. Cl. A61B 6/00; A61B 6/03; A61B 6/46; G06T 7/33

(52) U.S. Cl. CPC A61B 6/5241; A61B 6/03; A61B 6/463; A61B 6/466; G06T 7/337

(57) ABSTRACT

An X-ray CT apparatus and an image processing apparatus according to an embodiment include an acquisition unit, an alignment unit, and a setting unit. The acquisition unit acquires a data set in which a first image and a region of interest are paired from a database. The alignment unit aligns a second image with the first image of the acquired data set. The setting unit combines the region of interest set in the first image with the second image, and sets the region of interest in the second image.

### CROSS REFERENCE TO RELATED APPLICATIONS

This application is based upon and claims the benefit of priority from the prior Japanese Patent Application No. 2025-039739, filed on Mar. 12, 2025, the entire contents of which are incorporated herein by reference.

### FIELD

Embodiments described in the present specification and drawings relate to an X-ray CT apparatus, an image processing apparatus and an image processing method.

### BACKGROUND

When an image such as a medical image is analyzed, a region of interest (ROI) is frequently set. For example, in a clinical site, images are captured in various imaging fields of view, the ROI is set in each image, and subsequent analysis, diagnosis, and the like are performed.

Under such circumstances, conventionally, a method for automatically setting the ROI in the image has been developed. For automatic setting of the ROI, for example, a method of segmenting a tissue included in the ROI by a learning model and defining a region surrounding the tissue is known.

However, in a case where an image in which the ROI is desired to be set is different from an image used for learning of the learning model, segmentation is not appropriately performed, and a problem that the ROI cannot be correctly set may occur. Examples of the case where a problem that the ROI cannot be correctly set occurs in this way include a case where an imaging field of view of the image in which the ROI is desired to be set is different from an imaging field of view of the image used for learning.

### BRIEF DESCRIPTION OF THE DRAWINGS

FIG. 1 is a block diagram illustrating a configuration example of an image processing system according to a first embodiment.

FIG. 2 is a block diagram illustrating a configuration example of an image processing apparatus according to the first embodiment.

FIG. 3 is a flowchart illustrating an example of region of interest setting processing in the image processing system according to the first embodiment.

FIG. 4 is a diagram illustrating an example of a data set in the image processing system according to the first embodiment.

FIG. 5 is a diagram illustrating an example of a source image and a target image in the image processing system according to the first embodiment.

FIG. 6 is a diagram illustrating an example of voxelization of the target image in the image processing system according to the first embodiment.

FIG. 7 is a diagram illustrating an example of voxelization of the source image in the image processing system according to the first embodiment.

FIG. 8 is a diagram illustrating an example of alignment in the image processing system according to the first embodiment.

FIG. 9 is a diagram illustrating an example of setting of a region of interest in the image processing system according to the first embodiment.

FIG. 10 is a diagram illustrating an example of a first screen in the image processing system according to the first embodiment.

FIG. 11 is a diagram illustrating another example of the first screen in the image processing system according to the first embodiment.

FIG. 12 is a diagram illustrating an example of a second screen in the image processing system according to the first embodiment.

FIG. 13 is a block diagram illustrating a configuration example of the image processing apparatus according to a second embodiment.

FIG. 14 is a flowchart illustrating an example of the region of interest setting processing in the image processing system according to the second embodiment.

FIG. 15 is a diagram illustrating an example of the setting of the region of interest in the image processing system according to the second embodiment.

FIG. 16 is a diagram illustrating an example of the second screen in the image processing system according to the second embodiment.

FIG. 17 is a block diagram illustrating a configuration example of the image processing apparatus according to a third embodiment.

FIG. 18 is a flowchart illustrating an example of the region of interest setting processing in the image processing system according to the third embodiment.

FIG. 19 is a diagram illustrating an example of resampling processing in the image processing system according to the third embodiment.

FIG. 20 is a block diagram illustrating a configuration example of the image processing system according to a fourth embodiment.

FIG. 21 is a flowchart illustrating an example of the region of interest setting processing in an X-ray CT apparatus according to the fourth embodiment.

### DETAILED DESCRIPTION

Hereinafter, respective embodiments of the X-ray CT apparatus, the image processing apparatus and the image processing method will be described with reference to the accompanying drawings. In the embodiments below, the same reference signs are given for identical components in terms of configuration and function, and duplicate description is omitted.

### First Embodiment

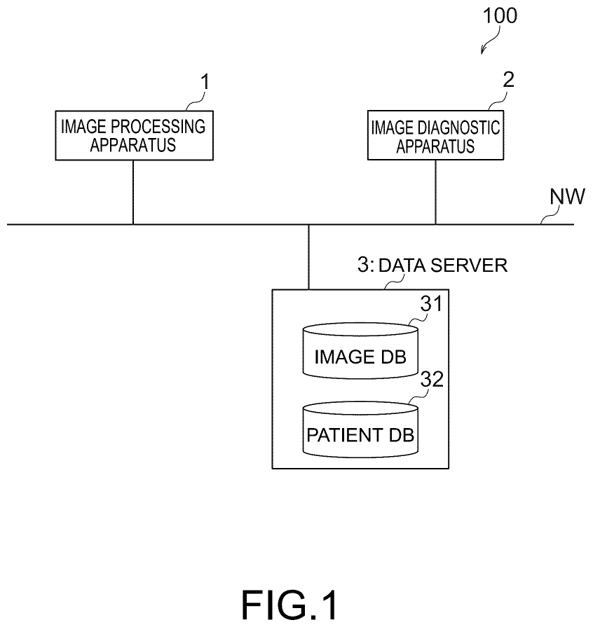

FIG. 1 is a block diagram illustrating a configuration example of an image processing system 100 according to a first embodiment. As illustrated in FIG. 1, the image processing system 100 according to the present embodiment includes an image processing apparatus 1, an image diagnostic apparatus 2, and a data server 3. The image processing apparatus 1, the image diagnostic apparatus 2, and the data server 3 are communicably connected to each other via a communication network NW.

The image processing apparatus 1 is an apparatus that processes an image such as a medical image. Specifically, the image processing apparatus 1 is an apparatus for setting a region of interest in the image. The image processing apparatus 1 is, for example, a workstation, an image interpretation terminal, and the like, and is operated by a user such as a doctor or a medical technician.

The image diagnostic apparatus 2 is an apparatus that generates an image by imaging a subject such as a patient (subject). The image diagnostic apparatus 2 is, for example, an X-ray diagnostic apparatus such as a general X-ray diagnostic apparatus or an X-ray computed tomography (CT) apparatus, or a medical image diagnostic apparatus such as a magnetic resonance imaging (MRI) apparatus, an ultrasonic diagnostic apparatus, a single photon emission computed tomography (SPECT) apparatus, or a positron emission computed tomography (PET) apparatus. The image diagnostic apparatus 2, which is the X-ray diagnostic apparatus, the MRI apparatus, or the ultrasonic diagnostic apparatus, images the subject and generates an X-ray image, an MRI image, or an ultrasound image, respectively. Note that in the present embodiment, a case where the image diagnostic apparatus 2 is the X-ray CT apparatus will be described as an example.

The data server 3 is a server that stores medical data and the like generated by the image processing apparatus 1 and the image diagnostic apparatus 2. For example, the data server 3 is an image server such as a picture archiving and communication system (PACS). Note that the data server 3 may be configured in the image processing apparatus 1 or may be provided on a cloud.

As illustrated in FIG. 1, in the present embodiment, the data server 3 includes an image DB (hereinafter, an image database) 31 and a patient DB (hereinafter, a patient database) 32.

The image database 31 stores a data set in which a target image and the region of interest are paired. The target image is an image in which the region of interest is set in advance. Such a target image may be captured by an apparatus such as the image diagnostic apparatus 2, may be acquired from the image diagnostic apparatus 2, may be acquired from a database in a hospital such as the PACS or a machine learning database, or may be acquired online, such as from the Internet. The machine learning database is, for example, a database used for learning of a learning model for segmentation. In the machine learning database, for example, an image and a mask for segmentation are stored in association with each other, and the corresponding image and the region of interest can be paired by setting the region of interest so as to surround the mask. The image database 31 corresponds to a database in the present embodiment. The target image corresponds to a first image in the present embodiment.

In the present embodiment, the image database 31 also stores data of a source image that is an image in which the region of interest is desired to be set. The source image is, for example, an image generated by imaging a patient in need of diagnosis by the image diagnostic apparatus 2. The source image corresponds to a second image in the present embodiment.

Note that each of the target image and the source image may have imaging condition information regarding imaging conditions as necessary. The imaging condition information includes, for example, imaging field of view information regarding an imaging field of view (FOV), an imaging angle, an imaging site, and the like, and information regarding a tube voltage, a tube current, a reconstruction function, a filter, a protocol name, resolution, and the like. The imaging condition information may be embedded in the target image and the source image, or may be stored in the image database 31 in association with the target image and the source image.

The patient database 32 stores various types of patient information (subject information) regarding the patient. The patient information includes, for example, physique information regarding a physique of the patient such as height, weight, abdominal circumference, and chest circumference, disease information regarding a disease name of the patient, and information regarding an age, sex, and the like of the patient. The patient database 32 may store electronic medical record information. Note that the patient information may be stored in the image database 31 in association with the target image and the source image. In this case, the patient database 32 may not be provided.

The communication network NW is used for communication among the image processing apparatus 1, the image diagnostic apparatus 2, and the data server 3. In the present embodiment, the communication network NW is an in-hospital network by a dedicated line in the hospital. Note that the communication network NW may be the Internet or the like.

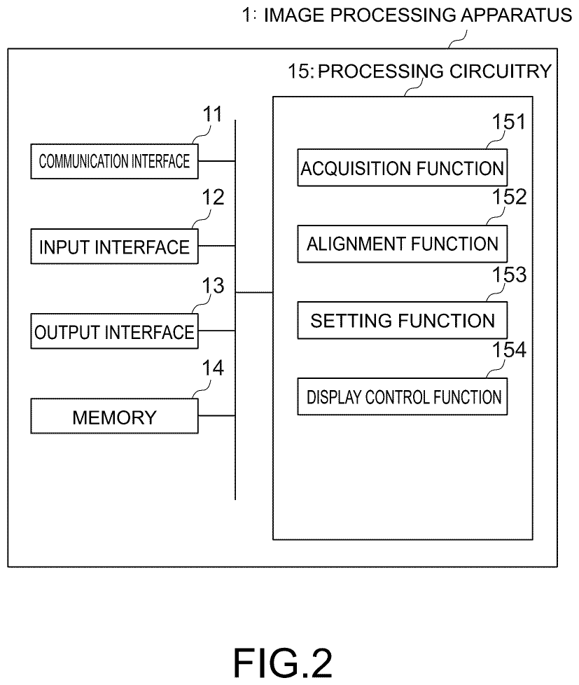

Next, the image processing apparatus 1 according to the present embodiment will be described in detail with reference to FIG. 2. FIG. 2 is a block diagram illustrating a configuration example of the image processing apparatus 1 according to the first embodiment. As illustrated in FIG. 2, the image processing apparatus 1 according to the present embodiment includes a communication interface 11, an input interface 12, an output interface 13, a memory 14, and a processing circuitry 15.

The communication interface 11 implements various communication protocols according to a form of the communication network NW. The communication interface 11 realizes communication with other devices via the communication network NW according to various communication protocols. In the present embodiment, the image processing apparatus 1 communicates with the image diagnostic apparatus 2 and the data server 3 via the communication interface 11.

The input interface 12 receives various input operations from an operator, converts the received input operations into electrical signals, and outputs the electrical signals to the processing circuitry 15. The input interface 12 includes, for example, a mouse, a keyboard, a trackball, a switch, a button, a joystick, a touch panel, and the like. The input interface 12 may be, for example, a user interface that receives a sound input such as a microphone. In a case where the input interface 12 is the touch panel, the input interface 12 may also have a display function of a display included in the output interface 13 described later.

Note that the input interface 12 is not limited to one including physical operating components such as the mouse or the keyboard. For example, an electric signal processing circuitry that receives an electric signal corresponding to an input operation from an external input device provided separately from devices and outputs the electric signal to a control circuit is also included in examples of the input interface 12.

The output interface 13 includes, for example, a display, a speaker, and the like. The display included in the output interface 13 corresponds to a display unit in the present embodiment. The display displays various types of information. For example, the display displays a screen generated by a display control function 154 of the processing circuitry 15, a graphical user interface (GUI) for receiving various input operations from the operator, and the like. The display is, for example, a liquid crystal display (LCD), a cathode ray tube (CRT) display, an organic electro luminescence (EL) display, or the like. The speaker outputs information input from the processing circuitry 15 as a sound.

The memory 14 is a non-transitory storage device that stores various types of information, and is realized by, for example, a random access memory (RAM), a semiconductor memory element such as a flash memory, a hard disk, an optical disk, or the like. For example, the memory 14 receives and stores various data collected by the processing circuitry 15. In addition, the memory 14 stores programs corresponding to various functions executed by circuits included in the image processing apparatus 1. Note that the memory 14 may be realized by a server group (cloud) connected to the image processing apparatus 1 via a network. In addition, various types of data handled in the present specification are typically digital data.

The processing circuitry 15 is a control circuit that performs overall control of the image processing apparatus 1, and is also an arithmetic circuit that performs various operations. In the present embodiment, the processing circuitry 15 includes an acquisition function 151, an alignment function 152, a setting function 153, and the display control function 154.

The acquisition function 151 acquires the data set in which the target image and the region of interest are paired from the database. In the present embodiment, the acquisition function 151 acquires the data set in which the target image and the region of interest are paired from the image database 31 of the data server 3. In the present embodiment, the data set acquired by the acquisition function 151 includes a plurality of pairs of the target image and the region of interest. The acquisition function 151 corresponds to an acquisition unit in the present embodiment.

In the present embodiment, the acquisition function 151 acquires the source image from the image database 31 of the data server 3. The source image is acquired based on, for example, an instruction of the user via the input interface 12. Note that the acquisition function 151 may acquire the source image from the image diagnostic apparatus 2 or from a portable storage medium such as a compact disc (CD), a digital versatile disc (DVD), or a flash memory instead of the data server 3.

The alignment function 152 aligns the source image with the target image of the data set acquired by the acquisition function 151. In the present embodiment, the alignment function 152 selects a pair to be used for alignment from a plurality of pairs included in the data set, and aligns the source image with the target image included in the selected pair. The alignment function 152 corresponds to an alignment unit in the present embodiment.

After the alignment by the alignment function 152, the setting function 153 combines the region of interest set in the target image with the source image, and sets the region of interest in the source image. The setting function 153 corresponds to a setting unit in the present embodiment.

For example, the display control function 154 displays the source image on which the region of interest is superimposed and displayed on the display included in the output interface 13. More specifically, the display control function 154 generates a screen including the source image on which the region of interest is superimposed and displayed, and causes the display to output the screen. The display control function 154 corresponds to a display control unit in the present embodiment.

Here, for example, processing functions executed by the acquisition function 151, the alignment function 152, the setting function 153, and the display control function 154, which are components of the processing circuitry 15 illustrated in FIG. 2, are recorded in the memory 14 in the form of a program executable by a computer. The processing circuitry 15 is, for example, a processor. A processor constituting the processing circuitry 15 reads out and executes the program from the memory 14 to realize a function corresponding to the read out program. In other words, the processing circuitry 15 in a state of reading out the program has the functions illustrated in the processing circuitry 15 of FIG. 2.

Note that FIG. 2 illustrates a case where the acquisition function 151, the alignment function 152, the setting function 153, and the display control function 154 are realized by the single processing circuitry 15, but the embodiment is not limited thereto. For example, the processing circuitry 15 may be configured by combining a plurality of independent processors, and the processors may respectively realize these functions by executing the programs. Further, the processing functions of the processing circuitry 15 may be realized by being appropriately distributed or integrated into a single or a plurality of processing circuitries.

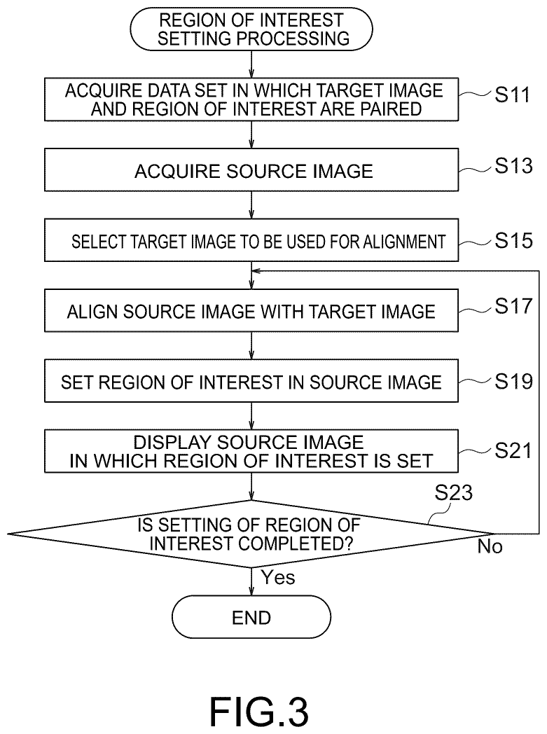

Next, an example of region of interest setting processing in the image processing system 100 configured as described above will be described with reference to FIG. 3. FIG. 3 is a flowchart illustrating an example of the region of interest setting processing in the image processing system 100 according to the first embodiment. The region of interest setting processing is, for example, processing executed when the user sets the region of interest in the source image using the image processing system 100.

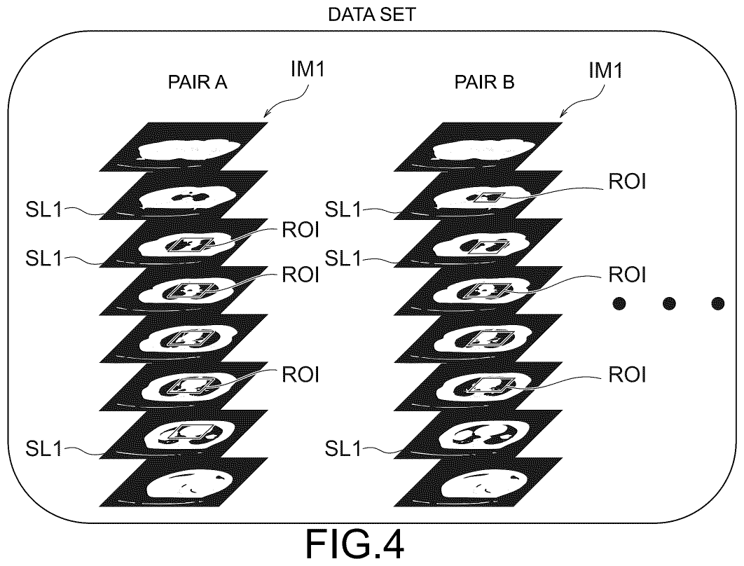

First, as illustrated in FIG. 3, the image processing apparatus 1 acquires a data set in which a target image IM1 and a region of interest ROI are paired (step S11). FIG. 4 is a diagram illustrating an example of the data set in the image processing system 100 according to the first embodiment. More specifically, as illustrated in FIG. 4, the acquisition function 151 of the image processing apparatus 1 acquires the data set in which the target image IM1 and the region of interest ROI are paired from the image database 31 of the data server 3.

In the present embodiment, the data set includes a plurality of pairs of the target image IM1 and the region of interest ROI. The region of interest ROI is set in advance for the target image IM1, and the set region of interest ROI is paired with the target image IM1. There are a plurality of such pairs of the target image and the region of interest, and the data set is formed by the plurality of pairs. In each pair included in the data set, at least one of the patient information and the imaging condition information may be different between the pairs, or both the patient information and the imaging condition information may be the same. For example, a pair A illustrated in FIG. 4 is an image obtained by imaging a periphery of a heart of a certain patient, and a pair B is an image obtained by imaging the periphery of the heart of a patient different from the patient. That is, the pair A and the pair B illustrated in FIG. 4 are different in at least patient information.

In the present embodiment, each target image IM1 included in each pair is a three-dimensional image including a plurality of slice images SL1. The region of interest ROI of each target image IM1 is set for each slice image SL1. That is, the region of interest ROI is set as a two-dimensional region in each slice image SL1. Note that instead of including the plurality of slice images SL1, each target image IM1 may be configured as one voxel data. In this case, in each target image IM1, the region of interest ROI may be set as a three-dimensional region in the voxel data.

The target image IM1 which is the three-dimensional image is an image captured by, for example, the X-ray CT apparatus or the MRI apparatus. Note that the target image IM1 is not limited to the three-dimensional image, and may be a two-dimensional image or a four-dimensional image. The target image IM1 which is the two-dimensional image is an image captured by, for example, a general X-ray imaging apparatus or the ultrasonic diagnostic apparatus. The target image IM1 which is the four-dimensional image is an image captured by, for example, a dynamic CT apparatus or the ultrasonic diagnostic apparatus.

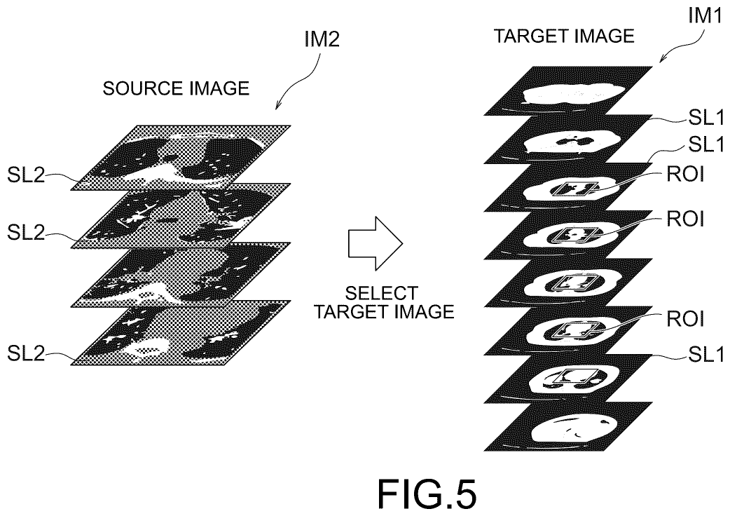

Next, as illustrated in FIG. 3, the image processing apparatus 1 acquires a source image IM2 (step S13). FIG. 5 is a diagram illustrating an example of the source image IM2 and the target image IM1 in the image processing system 100 according to the first embodiment. As illustrated in FIG. 5, the acquisition function 151 of the image processing apparatus 1 acquires data of the source image IM2 from the image database 31 of the data server 3. Note that this step S13 may be performed before step S11 of acquiring the data set.

In the present embodiment, similarly to the target image IM1, the source image IM2 is a three-dimensional image including a plurality of slice images SL2. Note that, similarly to the target image IM1, the source image IM2 may be the three-dimensional image configured as one voxel data, or may be the two-dimensional image or the four-dimensional image. In addition, in FIG. 5 and the subsequent drawings, a frame surrounding each slice image SL2 of the source image IM2 is illustrated for easy understanding of the drawings, but the data of the source image IM2 may not include information of this frame.

Next, as illustrated in FIG. 3, the image processing apparatus 1 selects the target image IM1 to be used for alignment (step S15). More specifically, as illustrated in FIG. 5, the alignment function 152 of the image processing apparatus 1 selects the target image IM1 similar to the source image IM2. In the present embodiment, the alignment function 152 selects a pair to be used for alignment from the data set based on at least one of the patient information regarding the patient in which each target image IM1 and the source image IM2 included in each pair of the data set are captured and the imaging condition information regarding imaging conditions of each target image IM1 and the source image IM2.

More specifically, in a case where the pair to be used for the alignment is selected from the data set based on the patient information, the alignment function 152 of the image processing apparatus 1 selects, for example, a pair including the target image IM1 in which the physique information regarding the physique of the patient out of the patient information is similar to the source image IM2 as the pair to be used for the alignment. Specifically, first, the alignment function 152 acquires physique information of the source image IM2. In addition, the alignment function 152 acquires physique information of each target image IM1 for each target image IM1 in the data set. Thereafter, the alignment function 152 selects a pair including the target image IM1 having physique information similar to the physique information of the source image IM2 from the data set. Note that the physique information of the target image IM1 and the physique information of the source image IM2 are similar to each other, for example, in a case where a ratio of height of patients whose target image IM1 and source image IM2 were captured is within 10%. In addition, “similar” in the present specification includes the same case. That is, the alignment function 152 may select a pair including the target image IM1 having the same physique information as the physique information of the source image IM2 from the data set. In addition, the physique information out of the patient information may be estimated by the image processing apparatus 1 based on pixel values of the target image IM1 and the source image IM2.

Note that, in a case where the pair to be used for the alignment is selected from the data set based on the patient information, the alignment function 152 of the image processing apparatus 1 may select, as the pair to be used for the alignment, a pair including the target image IM1 having the same disease information regarding the disease name out of the patient information as the source image IM2. Selection of the pair based on the disease information may be performed instead of selection of the pair based on the physique information or together with the selection of the pair based on the physique information.

On the other hand, in a case where the pair to be used for the alignment is selected from the data set based on the imaging condition information, the alignment function 152 of the image processing apparatus 1 selects, for example, a pair including the target image IM1 having the imaging field of view information regarding the imaging field of view similar to that of the source image IM2 out of the imaging condition information as the pair to be used for the alignment. Specifically, first, the alignment function 152 acquires the imaging field of view information of the source image IM2. In addition, the alignment function 152 acquires the imaging field of view information of each target image IM1 for each target image IM1 in the data set. Thereafter, the alignment function 152 selects a pair including the target image IM1 having the imaging field of view information similar to the imaging field of view information of the source image IM2 from the data set. Note that the target image IM1 and the source image IM2 have similar imaging field of view information, for example, in a case where the target image IM1 and the source image IM2 have the same imaging site and a difference in imaging angle is within 10%.

Note that, in a case where the pair to be used for alignment is selected from the data set based on the imaging condition information, the alignment function 152 of the image processing apparatus 1 may select, as the pair to be used for alignment, a pair including the target image IM1 having the imaging field of view including the imaging field of view of the source image IM2. In this case, the imaging field of view of the target image IM1 included in the selected pair includes the imaging field of view of the source image IM2.

Further, in a case of the pair to be used for the alignment is selected from the data set on based on both the patient information and the imaging condition information, the alignment function 152 of the image processing apparatus 1 selects, for example, a pair including the target image IM1 having patient information similar to the patient information of the source image IM2 and having imaging field of view information similar to the imaging condition information of the source image IM2 as the pair to be used for the alignment.

As another example, the alignment function 152 of the image processing apparatus 1 may select a pair including the target image IM1 having an image value similar to the pixel value of the source image IM2 as the pair to be used for the alignment. In this case, the alignment function 152 first calculates a matching degree between each target image IM1 and the source image IM2 based on the pixel value of each target image IM1 and the pixel value of the source image IM2 included in each pair of the data set. More specifically, the alignment function 152 acquires first pixel value information regarding the pixel value of each target image IM1 included in each pair of the data set and second pixel value information regarding the pixel value of the source image IM2. The pixel value is, for example, a luminance value of the target image IM1 and the source image IM2. Thereafter, the matching degree between the first pixel value information and the second pixel value information is calculated. The matching degree is calculated based on, for example, a difference between an average value of luminance values of the first pixel value information and an average value of luminance values of the second pixel value information. Alternatively, the matching degree may be calculated by binarizing the luminance values of the first pixel value information and the second pixel value information into white and black, and then comparing a ratio of a white portion to a black portion. Alternatively, the matching degree may be voxel similarity described later. After calculating the matching degree, the alignment function 152 selects a pair including the target image IM1 having the maximum matching degree among the pairs included in the data set as the pair to be used for the alignment.

Note that the alignment function 152 of the image processing apparatus 1 may select the pair to be used for the alignment by appropriately combining the patient information, the imaging condition information, and the matching degree. For example, the alignment function 152 selects, as the pair to be used for the alignment, a pair including the target image IM1 having a high matching degree with the source image IM2 among pairs including the target image IM1 in which both the patient information and the imaging condition information are similar to the source image IM2.

In the above example, the alignment function 152 of the image processing apparatus 1 selects one pair to be used for the alignment from among the pairs included in the data set. The present invention is not limited thereto, and the alignment function 152 may select a plurality of pairs to be used for alignment from among the pairs included in the data set. In this case, the pixel values of the target images IM1 included in the selected pairs may be averaged and used for alignment.

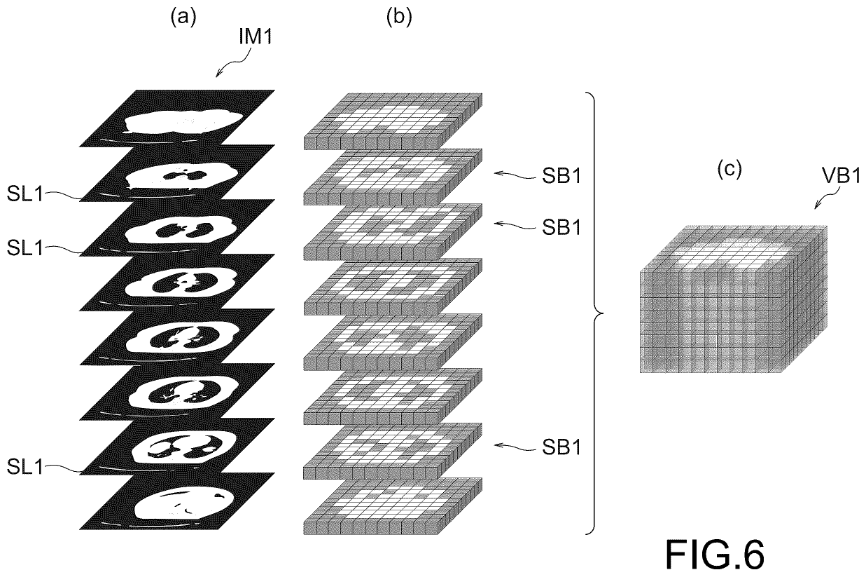

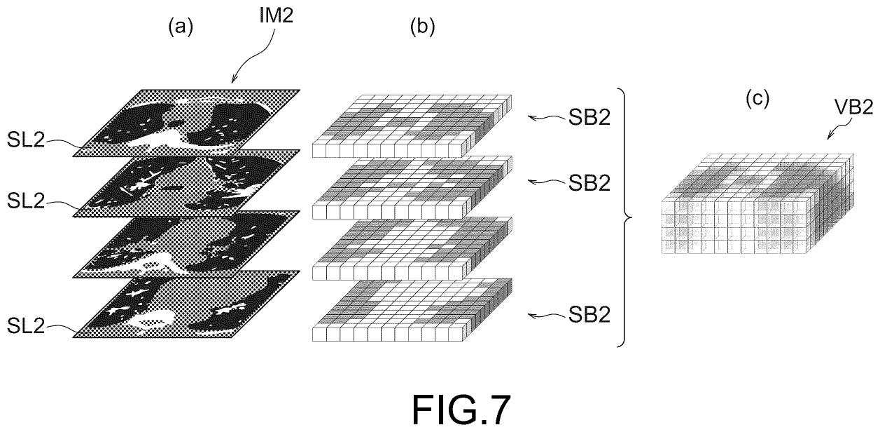

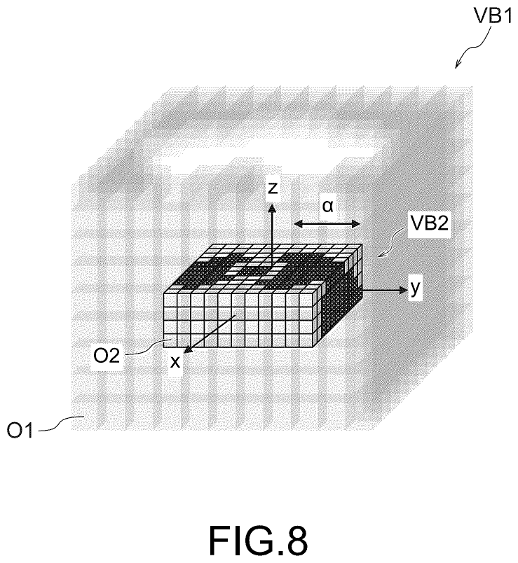

Next, as illustrated in FIG. 3, the image processing apparatus 1 aligns the source image IM2 with the target image IM1 (step S17). Hereinafter, this step will be described in detail with reference to FIGS. 6 to 8. FIG. 6 is a diagram illustrating an example of voxelization of the target image IM1 in the image processing system 100 according to the first embodiment. FIG. 7 is a diagram illustrating an example of voxelization of the source image IM2 in the image processing system 100 according to the first embodiment. FIG. 8 is a diagram illustrating an example of alignment in the image processing system 100 according to the first embodiment.

First, as illustrated in FIG. 6, the alignment function 152 of the image processing apparatus 1 voxelizes the target image IM1 to generate voxel data VB1. More specifically, the alignment function 152 generates voxel data SB1 for each slice illustrated in FIG. 6(b) using data of the slice image SL1 of the target image IM1 illustrated in FIG. 6(a). Each voxel data SB1 corresponds to, for example, data obtained by voxelizing pixel values of the corresponding slice image SL1. Thereafter, the alignment function 152 stacks the voxel data SB1 to generate the voxel data VB1 illustrated in FIG. 6(c). A value of each voxel of the voxel data VB1 corresponds to a first voxel value in the present embodiment.

Note that in the example of FIG. 6, the resolution of each voxel data SB1 is smaller than the resolution of each slice image SL1. The present invention is not limited thereto, and the resolution of each voxel data SB1 may be the same as the resolution of each slice image SL1.

Similarly, as illustrated in FIG. 7, the alignment function 152 of the image processing apparatus 1 voxelizes the source image IM2 to generate voxel data VB2. A method for generating the voxel data VB2 is similar to a method for generating the voxel data VB1 described above. That is, the alignment function 152 generates voxel data SB2 for each slice illustrated in FIG. 7(b) using data of the slice image SL2 of the source image IM2 illustrated in FIG. 7(a). Each voxel data SB2 corresponds to, for example, data obtained by voxelizing pixel values of the corresponding slice image SL2. Thereafter, the alignment function 152 stacks the voxel data SB2 to generate the voxel data VB2 illustrated in FIG. 7(c). A value of each voxel of the voxel data VB2 corresponds to a second voxel value in the present embodiment.

Thereafter, as illustrated in FIG. 8, the alignment function 152 of the image processing apparatus 1 performs the alignment based on a degree of similarity between the first voxel value for each voxel of the target image IM1 and the second voxel value for each voxel of the source image IM2. In the present embodiment, the alignment function 152 performs the alignment by searching for a position and a magnification that are the position and the magnification of the source image IM2 with respect to the target image IM1 and at which the degree of similarity between the first voxel value and the second voxel value is maximum. Hereinafter, this alignment will be described in detail.

First, the alignment function 152 of the image processing apparatus 1 generates an initial value (a seed value) of the position and the magnification. The initial value includes a coordinate value (x, y, z) representing the position and a value α representing the magnification. The initial value may be generated by a random number or may be generated as a prescribed value. Thereafter, the alignment function 152 temporarily sets, based on the initial value, the position and the magnification of a voxel O2 in the voxel data VB2 of the source image IM2 with respect to a voxel O1 in the voxel data VB1 of the target image IM1. Note that it is not limited to the position and the magnification of the voxel O2 with respect to the voxel O1, and the alignment function 152 may temporarily set the position and the magnification of the voxel different from the voxel O2 in the voxel data VB2 with respect to the voxel different from the voxel O1 in the voxel data VB1.

Thereafter, the alignment function 152 of the image processing apparatus 1 searches for the position and the magnification at which the degree of similarity between the voxel data VB1 and the voxel data VB2 is maximum. For example, first, the alignment function 152 calculates the degree of similarity between the voxel data VB1 and the voxel data VB2 at the temporarily set position and magnification. The degree of similarity is, for example, voxel similarity between a pixel value of each voxel of the voxel data VB1 and a pixel value of each voxel of the voxel data VB2 at the position and the magnification. The voxel similarity is calculated based on, for example, a cosine similarity between the voxel data VB1 and the voxel data VB2 in a region where the voxel data VB1 and the voxel data VB2 overlap each other in FIG. 8 or a value representing a correlation between other images. Thereafter, the alignment function 152 repeats changing the position and the magnification from the initial value and calculating the degree of similarity after the change, and determines the position and the magnification of the voxel O2 with respect to the voxel O1 in which the voxel similarity between the voxel data VB1 and the voxel data VB2 is maximum. Thus, the alignment of the source image IM2 with respect to the target image IM1 is completed.

Note that, for example, in a case where the target image IM1 and the source image IM2 are two-dimensional images, the alignment function 152 of the image processing apparatus 1 performs similar alignment using, for example, the voxel data SB1 and the voxel data SB2. In this case, the coordinate value indicating the position is, for example, (x, y). In a case where the target image IM1 and the source image IM2 are four-dimensional images, the alignment function 152 performs the alignment using a coordinate value (x, y, z, t) obtained by adding a value t representing time to the coordinate value (x, y, z) representing the position.

The alignment function 152 of the image processing apparatus 1 may perform a search by changing an angle of the source image IM2 with respect to the target image IM1 in addition to the position and the magnification of the source image IM2 with respect to the target image IM1. In this case, voxels may be cut again in order to match orientations of the voxels between the voxel data VB1 and the voxel data VB2.

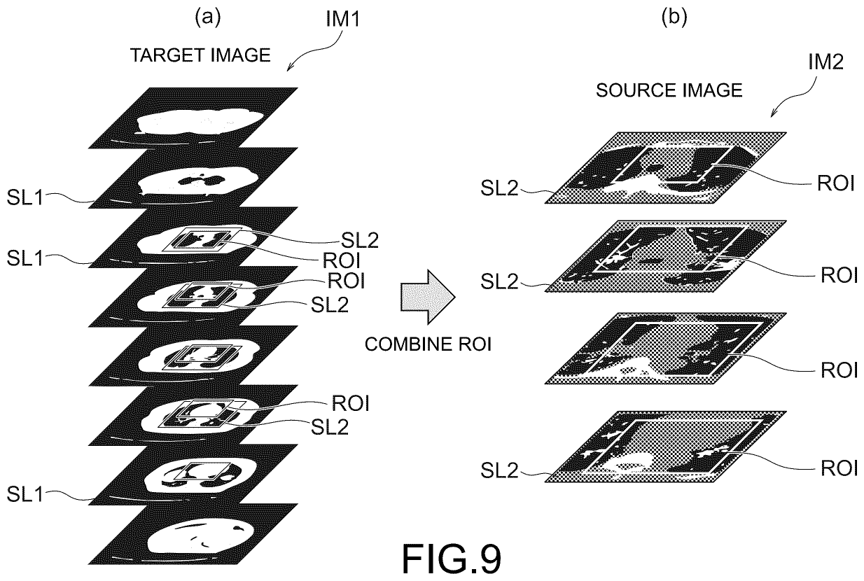

Next, as illustrated in FIG. 3, the image processing apparatus 1 sets the region of interest ROI in the source image IM2 (step S19). FIG. 9 is a diagram illustrating an example of setting of the region of interest ROI in the image processing system 100 according to the first embodiment. More specifically, as illustrated in FIG. 9(a), first, the setting function 153 of the image processing apparatus 1 superimposes the aligned source image IM2 on the target image IM1. Thereafter, as illustrated in FIG. 9(b), the setting function 153 combines the region of interest ROI set in each slice image SL1 of the target image IM1 with each slice image SL2 of the source image IM2 superimposed on the target image IM1. For example, the setting function 153 sets the region of interest ROI of each slice image SL2 aligned with each slice image SL1 at the same position as a position of the region of interest ROI set in each slice image SL1.

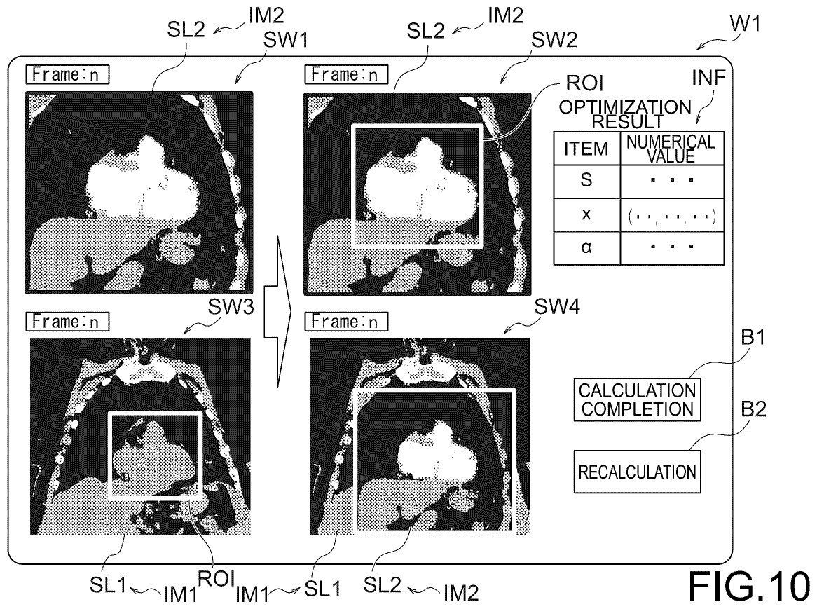

Next, as illustrated in FIG. 3, the image processing apparatus 1 displays the source image IM2 in which the region of interest ROI is set (step S21). FIG. 10 is a diagram illustrating an example of a first screen in the image processing system 100 according to the first embodiment. As illustrated in FIG. 10, the display control function 154 of the image processing apparatus 1 displays, for example, a first screen W1 on the display.

As illustrated in FIG. 10, for example, small screens SW1 to SW4 are displayed on the first screen W1. More specifically, the source image IM2 on which the region of interest ROI is not superimposed and displayed is displayed on the small screen SW1, the source image IM2 on which the region of interest ROI is superimposed and displayed is displayed on the small screen SW2, the target image IM1 on which the region of interest ROI is superimposed and displayed is displayed on the small screen SW3, and a composite image in which the source image IM2 is combined with a region corresponding to the source image IM2 in the target image IM1 is displayed on the small screen SW4. In the example of FIG. 10, in the small screens SW1 to SW4, the slice images SL1 and SL2 of the target image IM1 and the source image IM2 are respectively displayed as the target image IM1 and the source image IM2. In addition, “Frame: n” displayed at an upper left of the small screens SW1 to SW4 indicates that the slice images SL1 and SL2 displayed on the first screen W1 are nth slice images. Note that at least one of the small screens SW1, SW3, and SW4 may not be displayed on the first screen W1.

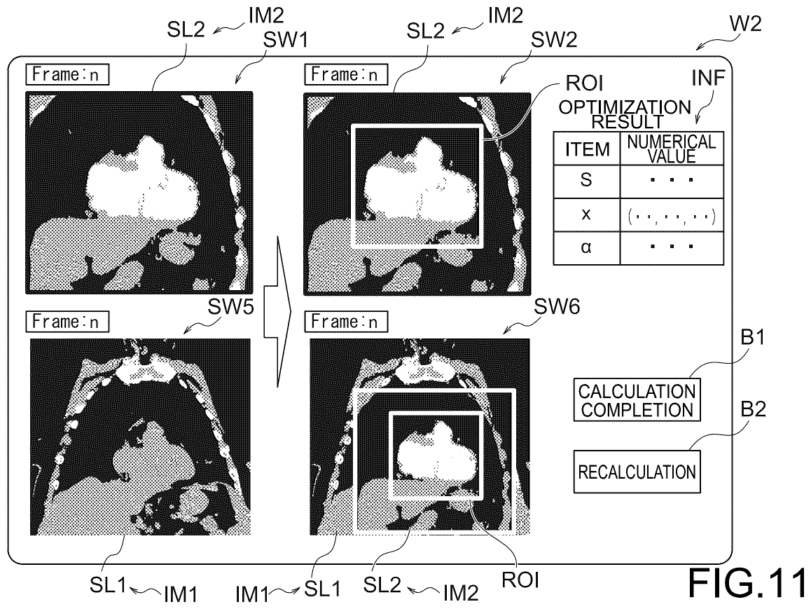

The display control function 154 of the image processing apparatus 1 may display a first screen W2 illustrated in FIG. 11 instead of the first screen W1 illustrated in FIG. 10. FIG. 11 is a diagram illustrating another example of the first screen in the image processing system according to the first embodiment.

As illustrated in FIG. 11, small screens SW5 and SW6 are displayed on the first screen W2 instead of the small screens SW3 and SW4. More specifically, the target image IM1 on which the region of interest ROI is not superimposed and displayed is displayed on the small screen SW5, and a composite image on which the region of interest ROI is superimposed and displayed is displayed on the small screen SW6. In the example of FIG. 11, in the small screens SW5 and SW6, the slice images SL1 and SL2 of the target image IM1 and the source image IM2 are respectively displayed as the target image IM1 and the source image IM2. Note that at least one of the small screens SW5 and SW6 may not be displayed on the first screen W2.

As described above, in the present embodiment, the display control function 154 of the image processing apparatus 1 displays the source image IM2 on which the region of interest ROI is superimposed and displayed on the display. Thus, the user can easily grasp the region of interest ROI set in the source image IM2. In addition, the display control function 154 displays, on the display, together with the source image IM2 on which the region of interest ROI is superimposed and displayed, at least one of the target image IM1 on which the region of interest ROI is not superimposed and displayed, the source image IM2 on which the region of interest ROI is not superimposed and displayed, the target image IM1 on which the region of interest ROI is superimposed and displayed, the composite image in which the source image IM2 is combined with a region corresponding to the source image IM2 in the target image IM1, and the composite image on which the region of interest ROI is superimposed and displayed. Thus, the user can easily grasp whether the region of interest ROI set in the source image IM2 is appropriate.

Note that, when the composite image of the small screen SW4 is displayed, the display control function 154 of the image processing apparatus 1 may generate image data of a replacement image in which a portion where image data of the source image IM2 is located in image data of the target image IM1 is replaced, and display the replacement image, or may simply display the source image IM2 on the target image IM1 in a superimposed manner.

As illustrated in FIGS. 10 and 11, alignment information INF is displayed on the first screens W1 and W2. The alignment information INF is information related to a result of the alignment by the alignment function 152 of the image processing apparatus 1. In the examples of FIGS. 10 and 11, the alignment information INF includes items S, X, and α and their numerical values. As the numerical value of the item S, the voxel similarity at the position and the magnification when the alignment is completed, that is, when the degree of similarity between the voxel data VB1 and the voxel data VB2 is maximum is indicated. As the numerical value of the item X, the coordinate value (x, y, z) when the alignment is completed is indicated. As the numerical value of the item α, the value α of the magnification when the alignment is completed is indicated. As described above, in the present embodiment, the display control function 154 of the image processing apparatus 1 displays, on the display, the alignment information INF regarding the result of the alignment together with the source image IM2 on which the region of interest ROI is superimposed and displayed. Thus, the user can easily grasp the result of the alignment. Note that the display control function 154 may not display the alignment information INF.

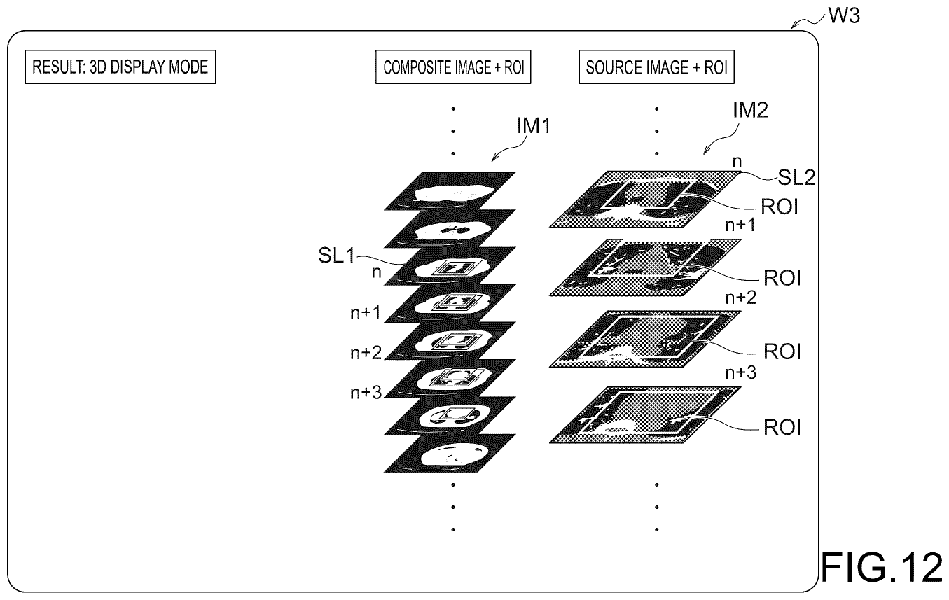

The display control function 154 of the image processing apparatus 1 may display a second screen W3 illustrated in FIG. 12 instead of the first screens W1 and W2 illustrated in FIGS. 10 and 11. FIG. 12 is a diagram illustrating an example of the second screen in the image processing system 100 according to the first embodiment.

As illustrated in FIG. 12, on the second screen W3, for example, a target image IM1 in which the source image IM2 is combined and the region of interest ROI is superimposed and displayed (that is, a composite image on which the region of interest ROI is superimposed and displayed), and the source image IM2 on which the region of interest ROI is superimposed and displayed are 3D displayed over a plurality of slice images. By displaying the second screen W3, the user can view the region of interest ROI set in each of the plurality of slice images SL2 at once. Note that in the second screen W3, each slice image SL2 of the source image IM2 may be displayed in association with each slice image SL2 of the target image IM1. For example, an nth slice image SL1 and an nth slice image SL2 may be displayed side by side on the second screen W3. Further, the slice images SL1 and SL2 may be scrolled in a vertical direction on the second screen W3. In these cases, the user can easily grasp a relationship between the slice images SL1 and SL2 and the region of interest ROI.

Note that the image processing apparatus 1 may receive an input for switching the display of the first screen W1 and the second screen W3 from the user. For example, when the user selects one of “Frame: n” on the first screen W1 illustrated in FIG. 10 via the input interface 12, the display control function 154 of the image processing apparatus 1 displays the second screen W3 illustrated in FIG. 12. Conversely, when the user selects one of the slice images SL1 and SL2 on the second screen W3 illustrated in FIG. 12, the display control function 154 displays the first screen related to the selected Frame. Specifically, when the user selects one of the nth slice images SL1 and SL2 on the second screen W3, the display control function 154 displays the first screen W1 related to the nth slice images SL1 and SL2 illustrated in FIG. 10.

Next, as illustrated in FIG. 3, the image processing apparatus 1 determines whether the setting of the region of interest is completed (step S23). In the present embodiment, the user looks at at least one of the first screens W1 and W2 and the second screen W3 and determines whether the setting of the region of interest is completed. For example, the user compares the small screens SW2 and SW3 of the first screen W1, and determines that the setting of the region of interest is completed when the region of interest ROI set in the source image IM2 is a region corresponding to the region of interest ROI set in the target image IM1. Alternatively, the user compares the small screens SW3 and SW4 of the first screen W1, and determines that the setting of the region of interest is completed when the source image IM2 is aligned at an appropriate position of the target image IM1.

In a case where it is determined that the setting of the region of interest is not completed (step S23: No), the image processing apparatus 1 continues the region of interest setting processing. In the present embodiment, the user who determines that the setting of the region of interest is not completed selects a button B2 on the first screen W1 via the input interface 12. The button B2 receives an input for recalculating the region of interest ROI from the user. The image processing apparatus 1 that receives the input determines that the setting of the region of interest is not completed. For example, the image processing apparatus 1 returns to step S17 and performs the alignment of the source image IM2 with respect to the target image IM1 again. Thereafter, the alignment function 152 of the image processing apparatus 1 changes the initial value of the position and the magnification, and searches for the position and the magnification at which the degree of similarity between the voxel data VB1 and the voxel data VB2 is maximum again.

On the other hand, in a case where it is determined that the setting of the region of interest is completed (step S23: Yes), the image processing apparatus 1 ends the region of interest setting processing. In the present embodiment, the user who determines that the setting of the region of interest is completed selects a button B1 on the first screen W1 via the input interface 12. The button B1 receives an input for completing calculation of the region of interest ROI from the user. The image processing apparatus 1 that receives the input determines that the setting of the region of interest is completed, and ends the region of interest setting processing illustrated in FIG. 3.

Through the above steps, the region of interest setting processing in the image processing system 100 according to the present embodiment ends. Note that step S23 may be omitted. In this case, after step S21, regardless of whether the result of the alignment is appropriate, the image processing apparatus 1 ends the region of interest setting processing.

As described above, according to the image processing apparatus 1 according to the present embodiment, after the source image IM2 is aligned with the target image IM1 in which the region of interest ROI is set, the region of interest ROI is combined with the source image IM2, and the region of interest ROI is set in the source image IM2. Thus, it is possible to realize automatic setting of the region of interest ROI without using segmentation. More specifically, in any of steps S11 to S23 of the region of interest setting processing, it is not necessary for the user to manually set the region of interest ROI.

Since the image processing apparatus 1 according to the present embodiment does not use segmentation, it is possible to avoid a problem that segmentation is not performed correctly and to reduce a cost for additional learning of the learning model for segmentation. Therefore, for example, in a case where the imaging field of view of the target image IM1 and the imaging field of view of the source image IM2 are different, it is possible to realize the automatic setting of the region of interest ROI robust to change in the imaging field of view without using segmentation.

In the image processing apparatus 1 according to the present embodiment, the data set includes the plurality of pairs of the target image IM1 and the region of interest ROI, and the alignment function 152 selects the pair to be used for the alignment based on at least one of the patient information and the imaging condition information. Thus, the alignment can be performed using a more appropriate target image IM1, and an alignment efficiency can be improved. In addition, the region of interest ROI can be automatically set for various source images IM2.

For example, the alignment function 152 of the image processing apparatus 1 selects a pair including the target image IM1 having physique information similar to that of the source image IM2 out of the patient information as the pair to be used for the alignment. In this case, it is possible to reduce an amount of calculation when searching for the magnification of the source image IM2 with respect to the target image IM1 in the alignment. Alternatively, searching for the magnification can be omitted. Alternatively, the alignment function 152 selects a pair including the target image IM1 having the same disease information as the source image IM2 out of the patient information as the pair to be used for the alignment. In this case, the target image IM1 can be easily selected.

Alternatively, the alignment function 152 of the image processing apparatus 1 selects the pair including the target image IM1 having the imaging field of view information similar to that of the source image IM2 out of the imaging condition information as the pair to be used for the alignment. In this case, it is possible to reduce an amount of calculation when searching for the position of the source image IM2 with respect to the target image IM1 in the alignment. Alternatively, the alignment function 152 selects the pair including the target image IM1 having the imaging field of view including the imaging field of view of the source image IM2 as the pair to be used for the alignment. In this case, the degree of similarity can be calculated more accurately at the time of the alignment.

Alternatively, the alignment function 152 of the image processing apparatus 1 calculates the matching degree between each target image IM1 and the source image IM2 based on the pixel value of each target image IM1 and the pixel value of the source image IM2 included in each pair of the data set, and selects the pair including the target image IM1 having the maximum matching degree as the pair to be used for the alignment. In this case, even when the target image IM1 having neither the patient information nor the imaging condition information or the source image IM2 having neither the patient information nor the imaging condition information is given, the pair to be used for the alignment can be appropriately selected.

### Second Embodiment

In the first embodiment described above, a determination as to whether the region of interest ROI set in the source image IM2 is appropriate may be added, and a warning may be displayed in a case where the region of interest ROI is not appropriate. Hereinafter, such a second embodiment will be described focusing on differences from the first embodiment.

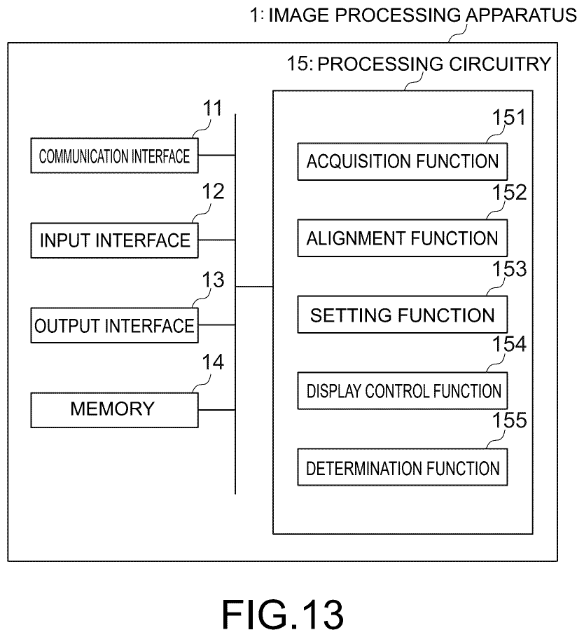

FIG. 13 is a block diagram illustrating a configuration example of the image processing apparatus 1 according to the second embodiment. As illustrated in FIG. 13, the processing circuitry 15 of the image processing apparatus 1 according to the present embodiment includes a determination function 155 in addition to units of the processing circuitry 15 of the image processing apparatus 1 according to the first embodiment.

The determination function 155 determines whether the region of interest ROI set in the source image IM2 by the setting function 153 is appropriate. In the present embodiment, the determination function 155 determines that the region of interest ROI set in the source image IM2 is appropriate when the region of interest ROI set in the source image IM2 is included in the imaging field of view of the source image IM2, and determines that the region of interest ROI set in the source image IM2 is not appropriate when the region of interest ROI is not included in the imaging field of view of the source image IM2. When it is determined that the region of interest ROI set in the source image IM2 is not appropriate, the display control function 154 displays a warning message on the display. The determination function 155 corresponds to a determination unit in the present embodiment.

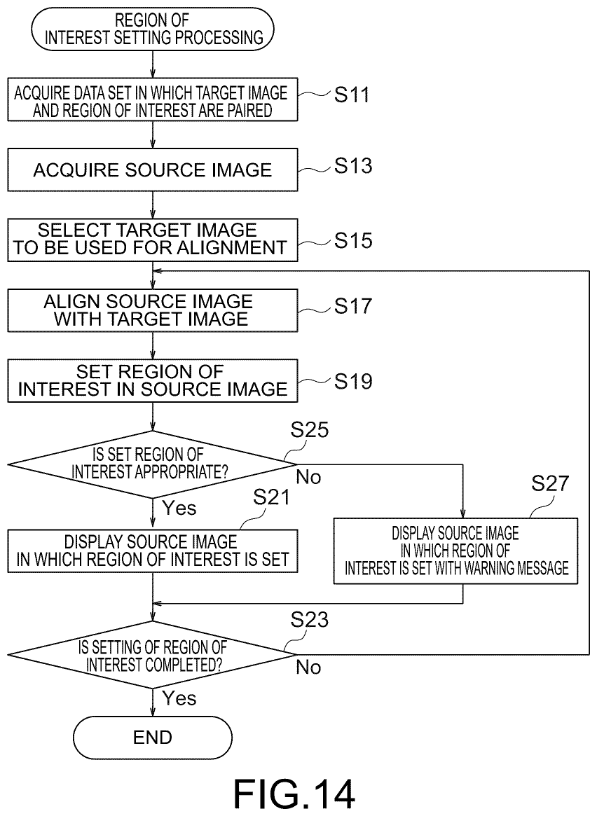

Next, an example of the region of interest setting processing in the image processing system 100 configured as described above will be described with reference to FIG. 14. FIG. 14 is a flowchart illustrating an example of the region of interest setting processing in the image processing system 100 according to the second embodiment.

As illustrated in FIG. 14, in the present embodiment, after setting the region of interest ROI in the source image IM2 in step S19, the image processing apparatus 1 determines whether the set region of interest ROI is appropriate (step S25).

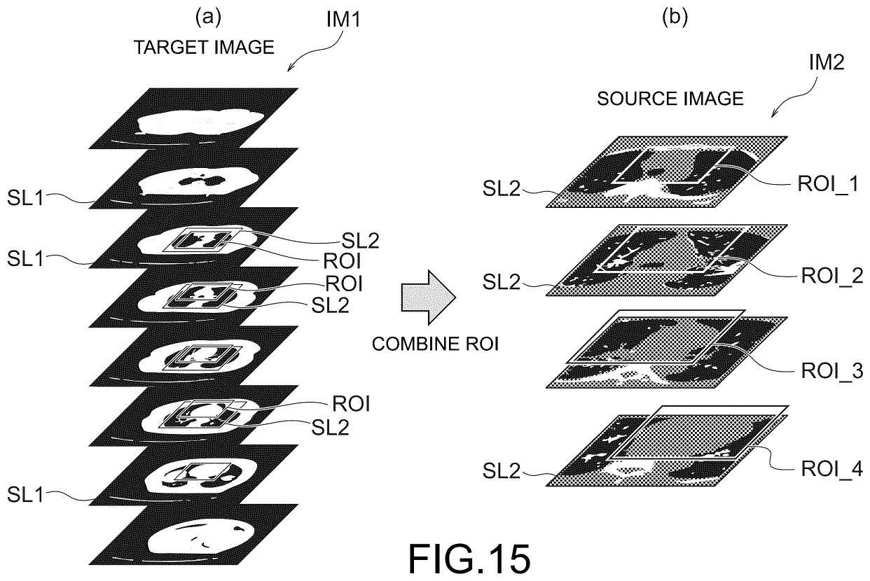

FIG. 15 is a diagram illustrating an example of the setting of the region of interest in the image processing system 100 according to the second embodiment. As illustrated in FIG. 15, the setting function 153 of the image processing apparatus 1 combines the region of interest ROI set in the target image IM1 with the source image IM2, and sets the region of interest ROI in the source image IM2. In the example of FIG. 15, a region of interest ROI_1, a region of interest ROI_2, a region of interest ROI_3, and a region of interest ROI_4 are respectively set as regions of interest ROI in the slice images SL2 of the source image IM2.

As illustrated in FIG. 15, each of the regions of interest ROI_1 and ROI_2 is included in the imaging field of view of the slice image SL2. On the other hand, the regions of interest ROI_3 and ROI_4 are outside the imaging field of view of the slice image SL2 and are not included in the imaging field of view of the slice image SL2. The determination function 155 of the image processing apparatus 1 determines that the regions of interest ROI_1 and ROI_2 are included in the imaging field of view of the source image IM2, and determines that the regions of interest ROI_3 and ROI_4 are not included in the imaging field of view of the source image IM2.

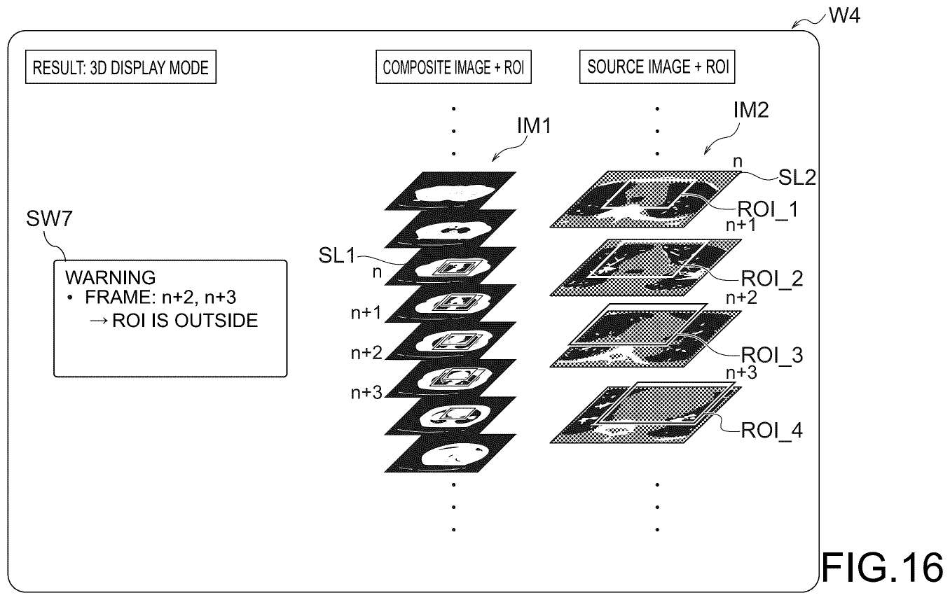

As illustrated in FIG. 14, when it is determined that the set region of interest ROI is not appropriate (Step S25: No), the image processing apparatus 1 displays the source image IM2 in which the region of interest ROI is set together with the warning message (step S27). FIG. 16 is a diagram illustrating an example of the second screen in the image processing system 100 according to the second embodiment. As illustrated in FIG. 16, the display control function 154 of the image processing apparatus 1 displays, for example, a second screen W4 including a small screen SW7. On the small screen SW7, characters “warning”, “Frame: n+2, n+3”, and “ROI is outside” are displayed as warning messages indicating that the set region of interest ROI is not appropriate. That is, in Frame: n+2 and Frame: n+3 corresponding to the slice images SL2 in which the regions of interest ROI_3 and ROI_4 are set, it is displayed that the region of interest ROI is outside the imaging field of view. After step S27, the image processing apparatus 1 performs step S23 as in the first embodiment.

On the other hand, when it is determined that the set region of interest ROI is appropriate (step S25: Yes), the image processing apparatus 1 performs steps S21 and S23 as in the first embodiment. Through the above steps, the region of interest setting processing in the image processing system 100 according to the present embodiment ends.

Note that when the determination function 155 of the image processing apparatus 1 determines that the alignment by the alignment function 152 is not appropriate (Step S25: No), the process may return to step S17, and the alignment may be automatically recalculated. In this case, the determination function 155 may determine that the alignment is not appropriately performed in a case where the degree of similarity between the target image IM1 and the source image IM2 in the alignment does not exceed a certain threshold. Thus, it is possible to suppress a possibility that a searched maximum value of the degree of similarity is not sufficiently large by selection of the initial value.

As described above, according to the image processing apparatus 1 according to the present embodiment, the user can easily grasp whether the setting of the region of interest ROI is completed.

### Third Embodiment

Next, a third embodiment in which image processing of the target image IM1 or the source image IM2 is performed before the alignment will be described focusing on differences from the second embodiment.

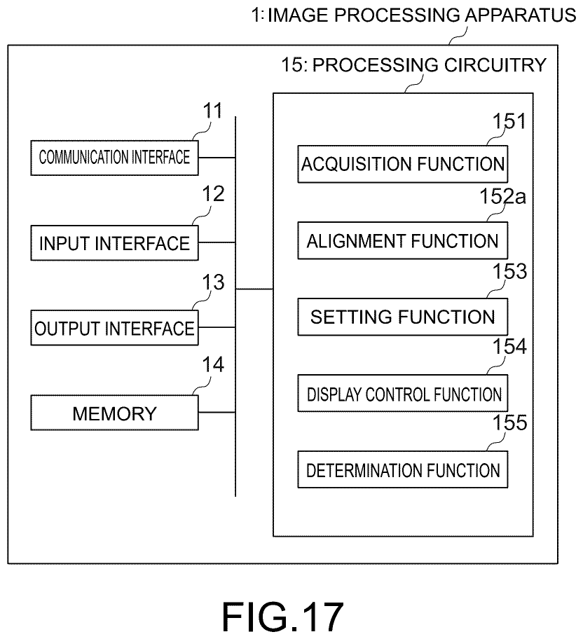

FIG. 17 is a block diagram illustrating a configuration example of the image processing apparatus 1 according to the third embodiment. As illustrated in FIG. 17, the processing circuitry 15 of the image processing apparatus 1 according to the present embodiment includes an alignment function 152a instead of the alignment function 152 of the processing circuitry 15 of the image processing apparatus 1 according to the second embodiment.

The alignment function 152a processes at least one of the target image IM1 and the source image IM2 by performing at least one of resampling processing and filtering processing in addition to the alignment function 152 described above. After processing at least one of the target image IM1 and the source image IM2, the alignment function 152a aligns the source image IM2 with the target image IM1.

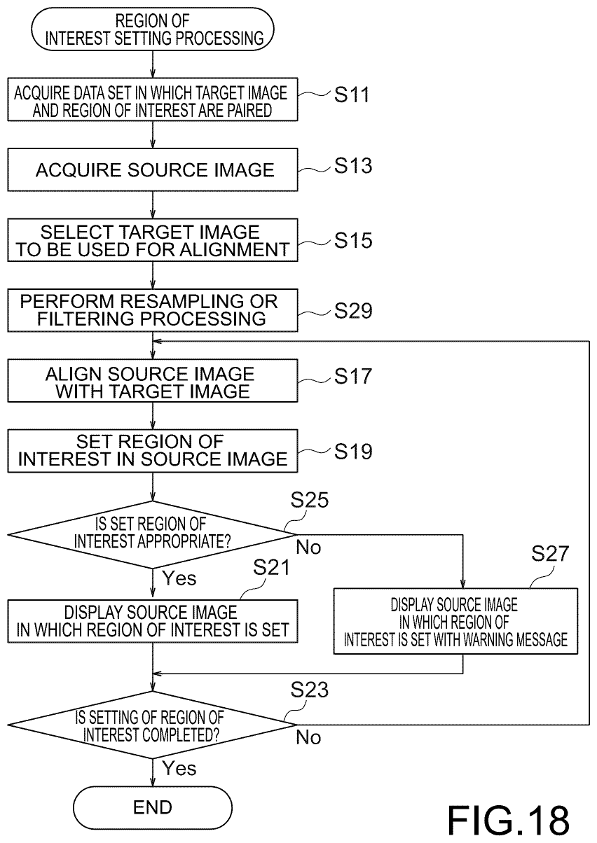

Next, an example of the region of interest setting processing in the image processing system 100 configured as described above will be described with reference to FIG. 18. FIG. 18 is a flowchart illustrating an example of the region of interest setting processing in the image processing system 100 according to the third embodiment.

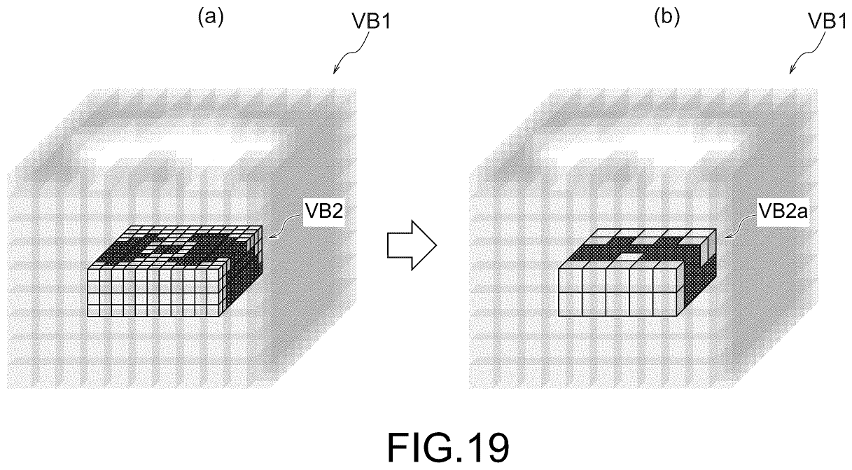

As illustrated in FIG. 18, after step S15, the image processing apparatus 1 performs the resampling processing or the filtering processing (step S29). FIG. 19 is a diagram illustrating an example of the resampling processing in the image processing system 100 according to the third embodiment. As illustrated in FIG. 19, the alignment function 152a of the image processing apparatus 1 performs, for example, down-sampling processing of the source image IM2 as the resampling processing of the source image IM2. More specifically, the voxel data VB2 of the source image IM2 illustrated in FIG. 19(a) has a higher resolution than the voxel data VB1 of the target image IM1. Therefore, it may be inefficient or difficult to calculate the degree of similarity by performing the alignment as it is. Therefore, as illustrated in FIG. 19(b), the alignment function 152a of the image processing apparatus 1 performs down-sampling processing of reducing resolution of the voxel data VB2 of the source image IM2 to generate voxel data VB2a.

In the example of FIG. 19(b), as a result of the down-sampling processing, the resolution of the voxel data VB2a matches the resolution of the voxel data VB1. Note that the alignment function 152a may perform up-sampling processing of increasing the resolution instead of the down-sampling processing. In place of the resampling processing of the source image IM2 or together with the resampling processing of the source image IM2, resampling processing of the target image IM1 may be performed.

The resampling processing may also be performed when the position and the magnification in the alignment are searched. Thus, it is possible to cope with a case where a position and a size of each voxel of the voxel data VB1 are different from a position and a size of each voxel of the voxel data VB2a when the position and the magnification are changed.

In addition, the alignment function 152a of the image processing apparatus 1 may perform the filtering processing on at least one of the target image IM1 and the source image IM2 instead of or together with the resampling processing. The filtering processing is, for example, smoothing processing using a Gaussian filter or the like, threshold processing such as binarization, trimming processing, or the like. The trimming processing is, for example, processing of removing a region sufficiently separated from the imaging field of view of the source image IM2 out of the target image IM1.

After step S27, the image processing apparatus 1 performs step S17 and subsequent steps as in the second embodiment. Through the above steps, the region of interest setting processing in the image processing system 100 according to the present embodiment ends.

As described above, according to the present embodiment, by performing the resampling processing or the filtering processing, the degree of similarity can be calculated more accurately at the time of the alignment.

Note that in the above description, the image processing apparatus 1 according to the present embodiment is obtained by replacing the alignment function 152 of the second embodiment with the alignment function 152a. The present embodiment is not limited to this, and can also be realized by replacing the alignment function 152 of the first embodiment with the alignment function 152a.

### Fourth Embodiment

Next, a fourth embodiment including an X-ray CT apparatus 4 as the image diagnostic apparatus 2 in the first embodiment will be described. Hereinafter, the fourth embodiment will be described focusing on differences from the first embodiment.

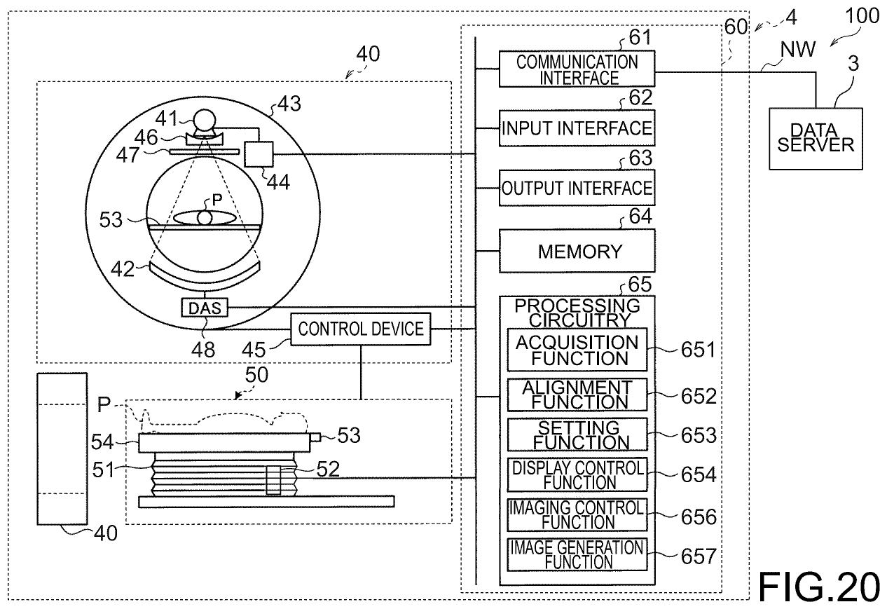

FIG. 20 is a block diagram illustrating a configuration example of the image processing system 100 according to a fourth embodiment. As illustrated in FIG. 20, the image processing system 100 according to the present embodiment includes the X-ray CT apparatus 4 which is one of image diagnostic apparatuses 2. In the present embodiment, the image processing apparatus 1 is incorporated in the X-ray CT apparatus 4 as a console device 60.

The X-ray CT apparatus 4 is an apparatus that generates an X-ray CT image by imaging a subject P using X-rays. The X-ray CT apparatus 4 includes, for example, a gantry device 40, a bed device 50, and the console device 60. Note that for convenience of description, a plurality of the gantry devices 40 are drawn in FIG. 20, but actually there is one gantry device 40.

The gantry device 40 includes an X-ray tube 41, an X-ray detector 42, a rotating frame 43, an X-ray high voltage device 44, a control device 45, a wedge 46, a collimator 47, and a data acquisition system (DAS) 48.

The X-ray tube 41 is a vacuum tube that generates X-rays by irradiating thermal electrons from a cathode (filament) toward an anode (target) by a high voltage from the X-ray high voltage device 44. The present embodiment is also applicable to a single tube type X-ray CT apparatus and a so-called multi-tube type X-ray CT apparatus in which a plurality of pairs of an X-ray tube and a detector are mounted on a rotation ring. Hardware that generates X-rays is not limited to the X-ray tube 41. For example, instead of the X-ray tube 41, the X-ray may be generated using a fifth generation system including a focus coil that focuses an electron beam generated from an electron gun, a deflection coil that electromagnetically deflects the electron beam, and a target ring that surrounds a half circumference of the subject P and generates X-rays by the deflected electron beam colliding therewith.

The X-ray high voltage device 44 includes an electric circuit such as a transformer and a rectifier, a high voltage generation circuit that generates a high voltage to be applied to the X-ray tube 41, and an X-ray control circuit that controls an output voltage according to the X-rays emitted from the X-ray tube 41. The high voltage generation circuit may be a transformer type or an inverter type. Note that the X-ray high voltage device 44 not only generates a high voltage, but also, for example, supplies power to the filament and supplies drive power when the anode is a rotary type. The X-ray high voltage device 44 may be provided on the rotating frame 43 or may be provided on a fixed frame side of the gantry device 40. Note that the fixed frame is a frame that rotatably supports the rotating frame 43.

The X-ray detector 42 detects the X-rays emitted from the X-ray tube 41 and passing through the subject P, and outputs a signal corresponding to a detected X-ray dose to the DAS 48. The X-ray detector 42 includes, for example, a plurality of X-ray detection element arrays in which a plurality of X-ray detection elements are arrayed in a channel direction (circumferential direction) along one arc centered on a focal point of the X-ray tube 41. The X-ray detector 42 has, for example, a structure in which a plurality of X-ray detection element arrays in which a plurality of X-ray detection elements are arrayed in the channel direction are arrayed in a slice direction (column direction or row direction).

The X-ray detector 42 is, for example, an indirect conversion type detector including a grid, a scintillator array, and an optical sensor array. The scintillator array includes a plurality of scintillators. The scintillator includes a scintillator crystal that outputs light of a photon amount corresponding to an incident X-ray dose. The grid includes an X-ray shielding plate that is disposed on a surface of the scintillator array on an X-ray incident side and absorbs scattered X-rays. Note that the grid may also be referred to as a collimator (a one-dimensional collimator or a two-dimensional collimator). The optical sensor array has a function of converting into an electric signal according to an amount of light from the scintillator, and includes, for example, an optical sensor such as a photodiode or a photomultiplier tube (PMT). Note that the X-ray detector 42 may be a direct conversion type detector having a semiconductor element that converts an incident X-ray into the electric signal.

The rotating frame 43 (frame base) is an annular frame that supports the X-ray tube 41 and the X-ray detector 42 facing each other and rotates the X-ray tube 41 and the X-ray detector 42 by the control device 45. For example, the rotating frame 43 is a casting made of aluminum. Note that the rotating frame 43 can further support the X-ray high voltage device 44 and the DAS 48 in addition to the X-ray tube 41 and the X-ray detector 42. Furthermore, the rotating frame 43 can further support various configurations not illustrated in FIG. 20. Hereinafter, in the gantry device 40, a portion that rotationally moves together with the rotating frame 43 and the rotating frame 43 are also referred to as a rotating portion. Note that although a description has been given of Rotate/Rotate-Type (third generation CT) in which the X-ray tube 41 and the X-ray detector 42 integrally rotate around the subject P, there are other various types such as Stationary/Rotate-Type (fourth generation CT) in which a large number of X-ray detection elements arrayed in a ring shape are fixed and only the X-ray tube 41 rotates around the subject P, and any type can be applied to the present embodiment.

Note that detection data generated by the DAS 48 is transmitted from a transmitter including a light emitting diode (LED) provided in the rotating frame 43 to a receiver including a photodiode provided in a non-rotating portion of the gantry device 40 by optical communication, and is transferred to the console device 60. Here, the non-rotating portion is, for example, a fixed frame or the like that rotatably supports the rotating frame 43. Note that a method of transmitting the detection data from the rotating frame 43 to the non-rotating portion of the gantry device 40 is not limited to optical communication, and any method may be adopted as long as data transmission can be performed between the rotating portion and the non-rotating portion.

The control device 45 includes a drive mechanism such as a motor and an actuator, and a circuit that controls the mechanism. The control device 45 receives an input signal from an input interface 62, an input interface provided in the gantry device 40, or the like, and controls operation of the gantry device 40 and the bed device 50. For example, the control device 45 controls rotation of the rotating frame 43, a tilt of the gantry device 40, operation of the bed device 50, and the like. As an example, the control device 45 rotates the rotating frame 43 about an axis parallel to an X-axis direction by input inclination angle (tilt angle) information as control to tilt the gantry device 40. Note that the control device 45 may be provided in the gantry device 40 or may be provided in the console device 60.

The wedge 46 is a filter for adjusting the X-ray dose emitted from the X-ray tube 41. Specifically, the wedge 46 is a filter that transmits and attenuates the X-ray emitted from the X-ray tube 41 such that the X-ray emitted from the X-ray tube 41 to the subject P has a predetermined distribution. For example, the wedge 46 is a wedge filter or a bow-tie filter, and is formed by processing aluminum or the like so as to have a predetermined target angle or a predetermined thickness.

The collimator 47 is a lead plate or the like for narrowing an irradiation range of the X-ray transmitted through the wedge 46, and forms a slit by a combination of a plurality of lead plates or the like. Note that the collimator 47 may also be referred to as an X-ray diaphragm. An opening degree and a position of the collimator 47 are adjusted by a collimator adjustment circuit (not illustrated). Thus, the irradiation range of the X-ray generated by the X-ray tube 41 is adjusted.

The DAS 48 includes an amplifier that amplifies the electric signal output from each X-ray detection element of the X-ray detector 42 and an A/D converter that converts the electric signal into a digital signal, and generates the detection data. The DAS 48 is implemented by, for example, a processor. The detection data generated by the DAS 48 is transferred to the console device 60.

The bed device 50 is a device that places and moves the subject P to be scanned. The subject P is, for example, a human patient or an animal. The bed device 50 includes a base 51, a bed driving device 52, a top plate 53, and a support frame 54. The base 51 is a housing that supports the support frame 54 so as to be movable in the vertical direction. The bed driving device 52 is a driving mechanism that moves the top plate 53 on which the subject P is placed in a long axis direction of the top plate 53. The top plate 53 is the plate on which the subject P is placed, and is disposed on an upper surface of the support frame 54. In the bed device 50 of the present embodiment, the top plate 53 is moved in the long axis direction of the top plate 53, so that the subject P placed on the top plate 53 can be moved between an imaging position where imaging by the gantry device 40 is performed and a retracted position retracted from the gantry device 40.

The console device 60 has substantially the same function as the image processing apparatus 1 in the first embodiment described above. Further, the console device 60 includes, for example, a communication interface 61, the input interface 62, an output interface 63, a memory 64, and a processing circuitry 65. The communication interface 61, the input interface 62, the output interface 63, the memory 64, and the processing circuitry 65 respectively correspond to the communication interface 11, the input interface 12, the output interface 13, the memory 14, and the processing circuitry 15 in the first embodiment described above, and have substantially equivalent functions.

The processing circuitry 65 includes an acquisition function 651, an alignment function 652, a setting function 653, and a display control function 654. The acquisition function 651, the alignment function 652, the setting function 653, and the display control function 654 respectively correspond to the acquisition function 151, the alignment function 152, the setting function 153, and the display control function 154 in the first embodiment described above, and have substantially equivalent functions.

In addition, the processing circuitry 65 includes an imaging control function 656 and an image generation function 657 in addition to the acquisition function 651, the alignment function 652, the setting function 653, and the display control function 654.

The imaging control function 656 images the subject P by controlling the X-ray tube 41 and the X-ray detector 42. More specifically, the imaging control function 656 controls the bed driving device 52 to move the subject P into an imaging port of the gantry device 40. The imaging control function 656 controls the X-ray high voltage device 44 to supply a high voltage to the X-ray tube 41. The imaging control function 656 adjusts the opening degree and the position of the collimator 47. The imaging control function 656 controls the control device 45 to rotate the rotating portion including the rotating frame 43. The imaging control function 656 causes the DAS 48 to collect projection data. Note that to reconstruct the CT image, projection data for 360° around the subject P is required, and projection data for 180° plus a fan angle is required in a half scan method. Any of reconstruction methods is applicable to the present embodiment. The imaging control function 656 corresponds to an imaging control unit in the present embodiment.

The image generation function 657 generates the X-ray CT image related to the subject P based on an output from the X-ray detector 42 by imaging performed by the imaging control function 656. In the present embodiment, the image generation function 657 generates the source image IM2 related to the subject P based on, for example, the output from the X-ray detector 42 by the imaging performed by the imaging control function 656. Note that the image generation function 657 may generate the target image IM1. The image generation function 657 generates data obtained by subjecting the detection data output from the DAS 48 to preprocessing such as logarithmic transformation processing, offset correction processing, inter-channel sensitivity correction processing, and beam hardening correction. Note that the data (detection data) before the preprocessing and the data after the preprocessing may be collectively referred to as the projection data. Further, for example, the image generation function 657 generates CT image data. Specifically, the image generation function 657 performs reconstruction processing using a filtered backprojection method, a successive approximation reconstruction method, or the like on the projection data after the preprocessing to generate the CT image data. The image generation function 657 converts the CT image data into tomographic image data of an arbitrary section or three-dimensional image data based on the input operation received from the operator via the input interface 62. The image generation function 657 corresponds to an image generation unit in the present embodiment.

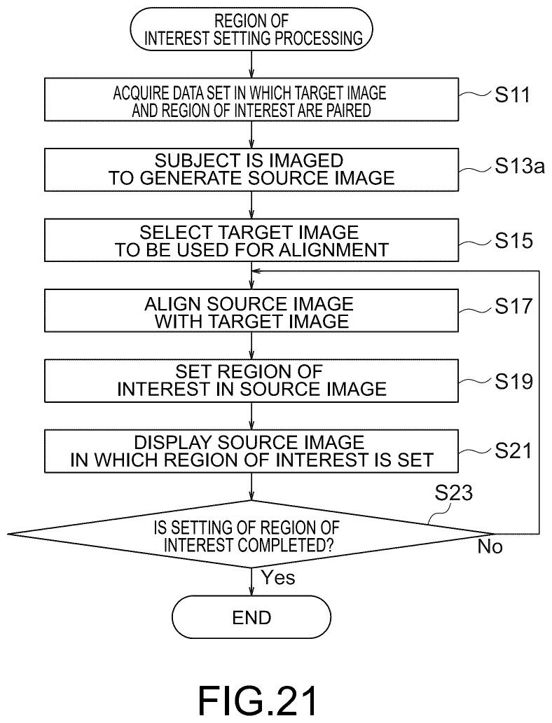

Next, an example of the region of interest setting processing in the image processing system 100 configured as described above will be described with reference to FIG. 21. FIG. 21 is a flowchart illustrating an example of the region of interest setting processing in the X-ray CT apparatus 4 according to the fourth embodiment.

As illustrated in FIG. 21, after step S11, the X-ray CT apparatus 4 images the subject P to generate the source image IM2 (step S13a). More specifically, first, the subject P is placed on the top plate 53 of the bed device 50 illustrated in FIG. 20. Thereafter, the imaging control function 656 of the processing circuitry 65 moves the top plate 53 with respect to the support frame 54. Thus, a site of the subject P to be imaged is inserted into the rotating frame 43. Thereafter, the imaging control function 656 of the processing circuitry 65 executes imaging of the subject P. Thereafter, the image generation function 657 generates the source image IM2 which is a captured image of the subject P. In the present embodiment, thereafter, the acquisition function 651 acquires the generated source image IM2. Step S15 and subsequent steps are the same as those in the first embodiment described above.

As described above, according to the image processing apparatus 1 according to the present embodiment, the region of interest ROI can be automatically set also for the source image IM2 captured by the X-ray CT apparatus 4. Thus, for example, inspection efficiency using the X-ray CT apparatus 4 can be improved.

Note that similarly to the second embodiment described above, the processing circuitry 65 of the X-ray CT apparatus 4 according to the present embodiment may further include a determination function having a function similar to the determination function 155. Furthermore, similarly to the third embodiment, the processing circuitry 65 of the X-ray CT apparatus 4 may include an alignment function having the same function as the alignment function 152a instead of the alignment function 652.

Note that the word “processor” used in above descriptions means circuits such as, for example, a Central Processing Unit (CPU), a Graphics Processing Unit (GPU), an Application Specific Integrated Circuit (ASIC), a programmable logic device (for example, a Simple Programmable Logic Apparatus (SPLD), a Complex Programmable Logic Apparatus (CPLD), and a Field Programmable Gate Array (FPGA)). The processor executes functions by reading and executing programs stored in the memory 14. Note that programs may be configured to be directly integrated in the processor instead of being storing in the memory 14. In this case, the processor realizes functions by reading and executing programs stored in the circuitry. Note that the processor is not limited to the case arranged as a single processor circuit, but may be configured as a single processor by combining a plurality of independent circuits to realize functions. Furthermore, a plurality of component elements in FIG. 2 may be integrated into one processor to realize the functions.

According to at least one embodiment described above, it is possible to realize the automatic setting of the region of interest without using segmentation.

While certain embodiments have been described, these embodiments have been presented by way of example only and are not intended to limit the scope of the inventions. The embodiments may be in a variety of other forms. Furthermore, various omissions, substitutions and changes may be made without departing from the spirit of the inventions. The embodiments and their modifications are included in the scope and the subject matter of the invention, and at the same time included in the scope of the claimed inventions and their equivalents.

1. An X-ray CT apparatus comprising:
an X-ray tube;
an X-ray detector configured to detect an X-ray emitted from the X-ray tube and passing through a subject; and
a processing circuitry configured to
acquire a data set in which a first image and a region of interest are paired from a database,
perform imaging of the subject by controlling the X-ray tube and the X-ray detector,
generate a second image related to the subject based on an output from the X-ray detector by the imaging,
align the second image with the first image of the acquired data set and
combine a region of interest set in the first image with the second image, and the region of interest is set in the second image.

2. An image processing apparatus comprising:
a processing circuitry configured to
acquire a data set in which a first image and a region of interest are paired from a database,
align a second image with the first image of the acquired data set; and
combine a region of interest set in the first image with the second image, and set the region of interest in the second image.

3. The image processing apparatus of claim 2.
wherein the data set includes a plurality of pairs of the first image and the region of interest, and
wherein the processing circuitry is further configured to select a pair to be used for the alignment from the data set based on at least one of subject information and imaging condition information regarding imaging conditions of each first image and the second image, wherein the subject information is related to a subject in which each first image included in each pair of the data set and the second image are captured.

4. The image processing apparatus of claim 3, wherein the processing circuitry is further configured to select a pair including a first image having physique information regarding a physique of the subject similar to the physique information of the second image out of the subject information as the pair to be used for the alignment.

5. The image processing apparatus of claim 3, wherein the processing circuitry is further configured to select a pair including a first image having imaging field of view information regarding an imaging field of view similar to the imaging field of view information of the second image out of the imaging condition information as the pair to be used for the alignment.

6. The image processing apparatus of claim 5, wherein the processing circuitry is further configured to select a pair including a first image having an imaging field of view including an imaging field of view of the second image as the pair to be used for the alignment.

7. The image processing apparatus of claim 3, wherein the processing circuitry is further configured to select a pair including a first image having disease information regarding a disease name the same as the disease information of the second image out of the subject information as the pair to be used for the alignment.

8. The image processing apparatus of claim 2,
wherein the data set includes a plurality of pairs of the first image and the region of interest, and
wherein the processing circuitry is further configured to
calculate a matching degree between each first image and the second image based on a pixel value of each first image included in each pair of the data set and a pixel value of the second image; and
select a pair including a first image having a maximum matching degree as a pair to be used for the alignment.

9. The image processing apparatus of claim 2, wherein the processing circuitry is further configured to display the second image on which the region of interest is superimposed and displayed on a display unit.

10. The image processing apparatus of claim 9,
wherein the processing circuitry is further configured to display, together with the second image on which the region of interest is superimposed and displayed, at least one of
the first image on which the region of interest is not superimposed and displayed,
the second image on which the region of interest is not superimposed and displayed,
the first image on which the region of interest is superimposed and displayed,
a composite image in which the second image is combined with a region corresponding to the second image in the first image, and
a composite image on which the region of interest is superimposed and displayed, on the display unit.

11. The image processing apparatus of claim 9,
wherein each of the first image and the second image includes a plurality of slice images, and
wherein the processing circuitry is further configured to display each slice image of the second image in association with each slice image of the first image on the display unit.

12. The image processing apparatus of claim 9, wherein the processing circuitry is further configured to display, on the display unit, alignment information regarding a result of the alignment together with the second image on which the region of interest is superimposed and displayed.

13. The image processing apparatus of claim 9,
wherein the processing circuitry is further configured to
determine whether the region of interest set in the second image is appropriate, and
display, when it is determined that the region of interest set in the second image is not appropriate, a warning message on the display unit.

14. The image processing apparatus of claim 2, wherein the processing circuitry is further configured to perform the alignment based on a degree of similarity between a first voxel value for each voxel of the first image and a second voxel value for each voxel of the second image.

15. The image processing apparatus of claim 14, wherein the processing circuitry is further configured to perform the alignment by searching for a position and a magnification that are the position and the magnification of the second image with respect to the first image and at which the degree of similarity is maximum.

16. The image processing apparatus of claim 2, wherein the processing circuitry is further configured to process at least one of the first image and the second image by performing at least one of resampling processing and filtering processing, and then perform the alignment.

17. An image processing method of setting a region of interest in a second image based on the region of interest set in a first image, the image processing method comprising:
acquiring a data set in which the first image and the region of interest are paired from a database;
aligning the second image with the first image of the acquired data set; and
setting the region of interest in the second image by combining the region of interest set in the first image with the second image.
